(19) United States
(12) Patent Application Publication
(10) Pub. No.: US 2020/0000058 A1
(43) Pub. Date: Jan. 2, 2020

(54) Method and apparatus that use the spectral reflectance characteristics of a reflector matrix, or other spectral filters, to limit available broad spectrum electromagnetic energy, reflected toward a patient target, to specific infrared bandwidths, to increase the effectiveness, comfort and/or duration of thermotherapy

(71) Applicant: Martin Allen McFarland, Boulder, CO (US)

(72) Inventor: Martin Allen McFarland, Boulder, CO (US)

(21) Appl. No.: 15/956,218

(22) Filed: Apr. 18, 2018

(51) Int. Cl. A01K 1/035; G02B 5/28; A61N 5/06

(52) U.S. Cl. CPC A01K 1/0353; E04B 2001/7691; A61N 5/06; G02B 5/282

(57) ABSTRACT

Reflected thermotherapy is a superior way to use ambient and available transmitted electromagnetic energy to treat a broad variety of illnesses. Warm blooded animals transmit a large amount of electromagnetic energy that can be reflected for thermotherapy. Using reflected electromagnetic energy from existing sources eliminates the need for a power supply, infrared lights, and cords. The problem is that in our modern energized world broad spectrum ambient and available transmitted electromagnetic energy from source may be contaminated with extra bandwidths of the full electromagnetic spectrum. This broad-spectrum energy when reflected onto a patient target can make the patient feel uncomfortably warm and produce undesirable effects. A variety of reflector coatings are available that can limit the spectra reflected back to a target from a broad spectra source. Gold finishes or tailored ceramic finishes are examples of spectral limiting coatings. When a gold reflector matrix is used, the spectral reflectance characteristics of gold limits the reflected energy to the infrared bandwidths. This reduces the hyperthermic discomfort of the patient target and allows the patient target to be treated with desired therapeutic infrared bandwidths for a much longer period of time. The gold reflector matrix can be attached to a fabric substrate. Once attached the substrate fabric, the resulting composite can be used to make a variety of apparatus. Blankets, beds, pads, athletic braces, and medicinal devices can all be lined with the gold composite fabric to achieve the desired therapeutic effect

### REFERENCES CITED

| | | |
|---|---|---|
| 9,423,538 | August 2016 | Tillotson et al. |
| 8,016,868 | September 2011 | Purcell |
| 7,766,950 | August 2010 | Castellani et al. |
| 6,007,245 | December 1999 | Looy |
| 4,925,743 | May 1990 | Ikeda et al. |
| 4,685,155 | August 1987 | Fingerhut et al. |
| 4,525,406 | June 1985 | Pollock |
| 4,508,776 | April 1985 | Smith |
| 4,433,019 | February 1984 | Chumbley |
| 4,420,521 | December 1983 | Carr |
| 4,401,707 | August 1983 | Bailey et al. |
| 4,395,455 | July 1983 | Frankosky |
| 4,186,294 | January 1980 | Bender |
| 4,030,646 | June 1977 | Switack et al. |

### BACKGROUND

### Field of the Invention

The present invention is directed to the treatment of people and animals with therapeutic and/or medicinal reflected infrared energy. A method is used to filter out undesirable reflected bandwidths to improve efficacy and the patients comfort. Because undesirable spectra are filtered out, the duration of the treatment can be extended without increasing hyperthermic side effects.

### Description of the Related Art

The treatment of illnesses with infrared energy is well established. In addition, the treatment of patients with reflected electromagnetic energy is also well established. The space blanket is a fitting example of the prior art. What has not been taught by the prior art is a method to filter or control the specific bandwidths reflected back to a target from available and ambient electromagnetic sources from a reflector matrix.

### SUMMARY

Reflected thermotherapy is a superior way to use ambient and available transmitted electromagnetic energy to treat a broad variety of illnesses. Warm blooded animals transmit a large amount of electromagnetic energy that can be reflected for thermotherapy. Using reflected electromagnetic energy from existing sources eliminates the need for a power supply, infrared lights, and cords. The problem is that in our modern energized world broad spectrum ambient and available transmitted electromagnetic energy from source may be contaminated with extra bandwidths of the full electromagnetic spectrum. This broad-spectrum energy when reflected onto a patient target can make the patient feel uncomfortably warm and produce undesirable effects. A variety of reflector coatings are available that can limit the spectra reflected back to a target from a broad spectra source. Gold finishes or tailored ceramic finishes are examples of spectral limiting coatings. When a gold reflector matrix is used, the spectral reflectance characteristics of gold limits the reflected energy to the infrared bandwidths. This reduces the hyperthermic discomfort of the patient target and allows the patient target to be treated with desired therapeutic infrared bandwidths for a much longer period of time. The gold reflector matrix can be attached to a fabric substrate. Once attached the substrate fabric, the resulting composite can be used to make a variety of apparatus. Blankets, beds, pads, athletic braces, and medicinal devices can all be lined with the gold composite fabric to achieve the desired therapeutic effect.

### BRIEF DESCRIPTION OF THE DRAWINGS

FIG. 1 illustrates the ambient broad spectrum electromagnetic energy that is radiating onto a dog bed from outside sources.

FIG. 2 illustrates the electromagnetic energy being transmitted from a dog at rest.

FIG. 3 illustrates the combination of ambient, transmitted, and reflected electromagnetic energy related to a dog sleeping on the bed shown in FIG. 1.

FIG. 4 illustrates a dog sleeping comfortably on the bed shown in FIG. 1 while being treated with reflected infrared.

### DETAILED DESCRIPTION

Everything that has mass in our world absorbs, reflects and transmits electromagnetic energy. This electromagnetic energy is classified in a spectrum that ranges from gamma rays, through the visible light spectrum, infrared spectrum, and ending with extremely low frequency energy. Many bandwidths of the electromagnetic spectrum have been used for treating illness and improving health. From the moment man warmed himself in front of a fire, a long history of using the benefits of electromagnetic energy began.

Many different frequencies of the electromagnet spectrum have been used to treat a broad variety of illnesses. Although electromagnetic frequencies affect the body in fundamentally the same way, the body responds or reacts in a unique way for each frequency. For targeted, or managed treatment, with infrared energy it is necessary to be able to generate specific frequencies that produce a beneficial physiological response or medical effect. This science is well established, and devices exist to produce a broad variety of frequencies. Infrared has been shown to cause increased circulation, cellular profusion, and cellular metabolism. It has also been shown to stimulate the nerves. The increased circulation and profusion assist the body with detoxification. Our research has shown that reflected infrared is also antimicrobial and helps a variety of skin illness issues.

Reflected thermotherapy is a superior way to use available ambient and available transmitted electromagnetic energy to treat a broad variety of illnesses. The surface of the reflector can be shaped in a way to focus the reflected energy for specific treatment, or to disperse the reflected energy into a wide treatment area. Using reflected electromagnetic energy from existing sources eliminates the need for a power supply, infrared lights, and cords.

Warm blooded animals transmit a large amount of electromagnetic energy that can be used for therapy. At any time, our environment is filled with sun light, microwaves, visible light, radio waves, infrared waves, etc. When using reflected electromagnetic energy, the need exists to be able to control the reflected energy frequencies to produce the desired healing or health results.

The problem is that, in our modern energized world, broad spectrum ambient and available transmitted electromagnetic energy may contain any bandwidth of the full electromagnetic spectrum. This broad-spectrum energy when reflected onto a target patient can make the patient feel uncomfortably warm and produce undesirable effects.

Even the bandwidth of electromagnetic energy given off by a living body can be reflected back to the body in specific spectra. This filtering by the reflector offers the opportunity to target specific cellular components or body processes with reflected therapeutic energy.

A variety of reflector coatings are available that can limit the spectra that is reflected back to a target from a broad spectra source. Gold finishes or tailored ceramic finishes are examples of spectral limiting coatings. Spectral limiting reflector finishes can be tailored to limit upper, mid, and lower ranges from an electromagnetic source. When a gold coated reflector matrix is used, the spectral reflectance characteristics of gold limits the reflected energy to the infrared bandwidths. This effectively reduces the discomfort of the target patient caused by higher frequency bandwidths and allows the patient to be treated with desired therapeutic infrared bandwidths for a much longer period. Tailoring the bandwidths that are reflected to the patient can significantly increase healing efficacy and reduce undesirable side effects.

The aforementioned gold reflector matrix can be attached to a fabric substrate. Once attached, the composite fabric can be used to make a variety of therapeutic apparatuses. These can include, blankets, beds, back braces, knee bracing covers, elbow bracing covers, etc. The potential applications include both human and animal patients.

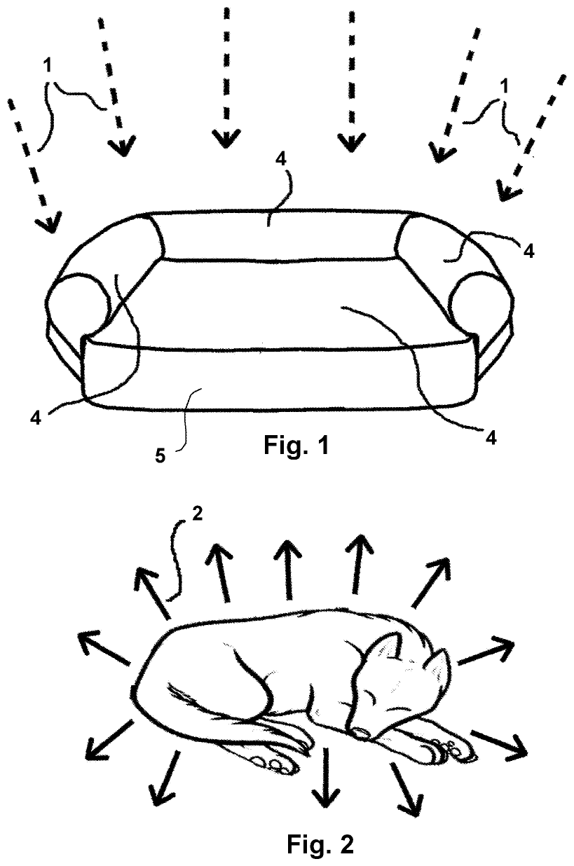

One apparatus that can be made using a gold reflector matrix fabric is a dog bed. FIG. 1 shows a dog bed 5. The gold reflector matrix fabric covers surface 4. Arrows 1 represent broad spectrum ambient electromagnetic energy that is affecting the bed 5.

FIG. 2 shows a dog at rest on any typical surface. Arrows 2 represent the electromagnetic energy being transmitted from the dog. These include visible and infrared energy.

FIG. 3 shows arrows representing all the different wavelengths affecting a dog at rest on bed 5. Arrow 1 is the ambient broad spectrum electromagnetic energy. Arrow 2 represents the electromagnetic energy being transmitted by the dog. Arrow 3 represents the limited spectrum infrared energy being reflected to the dog by the gold reflector fabric substrate that is covering the surfaces 4 (from FIG. 1) of the dog bed.

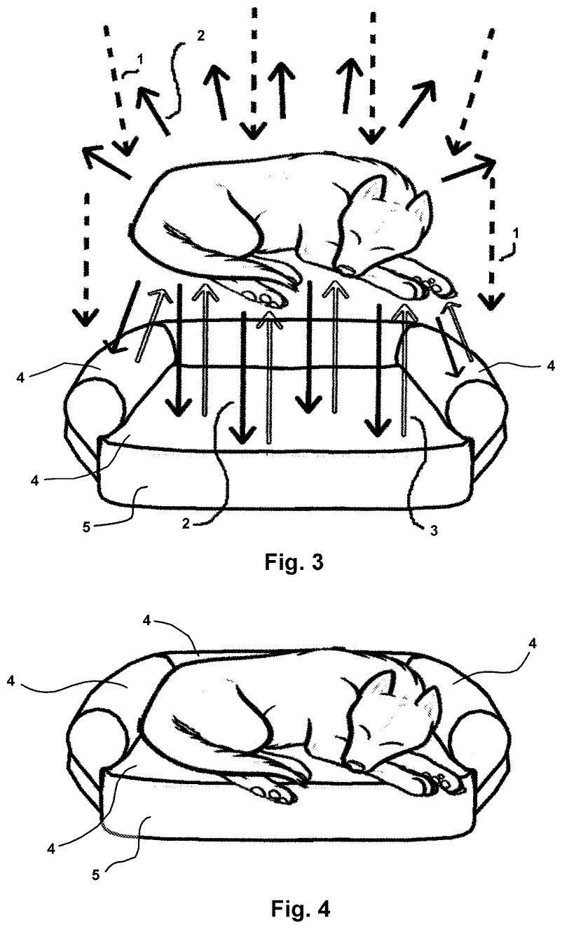

FIG. 4 is the dog resting peacefully on dog bed 5, being comfortably treated by the limited spectrum reflected infrared energy from surface 4 while he sleeps. For the dog to get the most benefit from the reflected infrared he needs to be comfortable and may have to remain in place for several hours. If electromagnetic energy from all the sources shown in FIG. 3 are reflected toward the dog he could be overexposed. As a result of the overexposure, the dog will feel uncomfortable and not stay on the bed. The bed may also contain additional structures to allow the gold reflector matric fabric to breath. Currently this structure is a poly fiber mesh that allows cooling air to convect under and through the gold reflector matrix fabric. In addition, the bed would also be designed with a way to turn off the reflected therapy effect. Currently the opposite side of the bed surface is a breathable fabric, and if it is necessary to shut off the reflected energy; you simply turn the bed surfaces over and let the dog sleep on the other side of the bed

The gold reflector matrix fabric can also be used to make a bed pad or kennel pad. The principles would be the same. The upper surface of the bed pad is the gold reflector matrix fabric. There is a breathable sub structure for padding and cooling convection. The back side of the bed pad is a breathable material that shuts off the reflected therapy effect when the bed is flipped over, with the reflective side down.

The gold reflector matrix fabric can also be used to make blankets. One side of the blanket would be the gold reflector fabric and the rest of the blanket would be made of standard blanket materials. The reflected therapy effect can be shut off by removing the blanket or flipping it over. A smaller blanket can be used to apply the reflected therapy effect to a specific area of the dog or human patient. It can also be used to cover existing bedding surfaces from other suppliers to allow the user to keep a previous dog bed and still be able to use the reflected therapy.

The gold reflector fabric matrix can also be used in athletic braces, pads and supports. The gold reflector fabric matrix can be bonded to a neoprene substrate to make it durably stretchy. This material can be used to make knee, back, wrist, shoulder, ankle, etc. braces. The gold reflector would be on the inside to reflect transmitted infrared energy back to the source. To shut off the effect you would simply remove the device.

The gold reflector fabric can be used to make a broad variety of medicinal devices in human care facilities. It can be used to make a bed sheet to treat patients, who spend long time periods in bed, with the healing effects of passive infrared. Infrared has been shown to improve skin health and detoxification. To shut off the effect you would simply remove the sheet. The ability to apply reflected infrared therapy over an extended period of time comfortably may could be used to treat, cartilage issues caused by poor joint profusion. It could be used to treat any number neurological issues. It could be used to treat a broad variety of skin issues. It can also be used to treat a variety of muscular issues.

I claim:

1) A method for using the spectral reflectance characteristics of a gold reflector matrix, to limit broad spectrum electromagnetic energy, reflected toward a patient target, to infrared bandwidths, to increase the efficacy, duration and/or comfort of thermotherapy.

2) An apparatus that uses the spectral reflectance characteristics of a gold reflector matrix, to limit broad spectrum electromagnetic energy, reflected toward a patient target, to infrared bandwidths, to increase the efficacy, duration and/or comfort of thermotherapy.

3) A system for using the spectral reflectance characteristics of a gold reflector matrix, to limit broad spectrum electromagnetic energy, reflected toward a patient target, to infrared bandwidths, to increase the efficacy, duration and/or comfort of thermotherapy.

4) A method for using the spectral reflectance characteristics of a reflector matrix, to limit broad spectrum electromagnetic energy, reflected toward a target, to specific bandwidths, to increase the efficacy, duration and/or comfort of thermotherapy.

5) An apparatus that uses the spectral reflectance characteristics of a reflector matrix, to limit broad spectrum electromagnetic energy, reflected toward a target, to specific bandwidths, to increase the efficacy, duration and/or comfort of thermotherapy.

6) A system for using the spectral reflectance characteristics of a reflector matrix, to limit broad spectrum electromagnetic energy, reflected toward a target, to specific bandwidths, to increase the efficacy, duration and/or comfort of thermotherapy.
